(19) United States
(12) Patent Application Publication
(10) Pub. No.: US 2022/0248964 A1
(43) Pub. Date: Aug. 11, 2022

(54) BLOOD PRESSURE MEASURING APPARATUS AND NON-TRANSITORY RECORDING MEDIUM STORING CONTROL PROGRAM

(71) Applicant: OMRON HEALTHCARE Co., Ltd., Kyoto (JP)

(72) Inventors: Daisuke ISHIHARA, Kyoto (JP); Miki OKUGAWA, Kyoto (JP); Motofumi NAKANISHI, Kyoto (JP); Yukiko MITSUNAMI, Kyoto (JP)

(21) Appl. No.: 17/660,564

(22) Filed: Apr. 25, 2022

Related U.S. Application Data
(63) Continuation of application No. PCT/JP2020/036914, filed on Sep. 29, 2020.

(30) Foreign Application Priority Data
Oct. 29, 2019 (JP) 2019-196628

(51) Int. Cl. A61B 5/021; A61B 5/00

(52) U.S. Cl. CPC A61B 5/021; A61B 5/741; A61B 5/742; A61B 5/7455

(57) ABSTRACT

Provided is a technique capable of prompting execution of highly useful blood pressure measurement. That is, the blood pressure measuring apparatus according to an aspect of the present invention is a blood pressure measuring apparatus for performing blood pressure measurement, the blood pressure measuring apparatus including a notification unit that performs a notification to prompt a user to perform the blood pressure measurement, a time setting unit capable of setting a blood pressure measurement time by an instruction from the user, a storage unit that stores a recommended time period of the blood pressure measurement, and a control unit that performs control to cause the notification unit to start the notification based the blood pressure measurement time set by the time setting unit, and cause the notification unit to stop the notification when the recommend time period passes.

### CROSS-REFERENCE TO RELATED APPLICATIONS

This application is the U.S. national stage application filed pursuant to 35 U.S.C. 365(c) and 120 as a continuation of International Patent Application No. PCT/JP2020/036914, filed Sep. 29, 2020, which application claims priority to Japanese Patent Application No. 2019-196628, filed Oct. 29, 2019, which applications are incorporated herein by reference in their entireties.

### TECHNICAL FIELD

The present invention relates to a blood pressure measuring apparatus and a non-transitory recording medium storing a control program.

### BACKGROUND ART

A known health appliance use guidance apparatus sets a time period in which a user uses a health appliance, and performs a notification every day when the set time period comes (Patent Literature (PTL) 1). In addition, a known biological information measuring apparatus notifies the user that a current time is included in a predetermined time period including a scheduled measurement time by blinking of a light emitter or a display (PTL 2). In addition, a known a blood pressure measuring apparatus generates an alarm for prompting a patient to measure blood pressure every morning and night (PTL 3).

### CITATION LIST

### Patent Literature

PTL 1: JP H11-178798 A
PTL 2: JP 2008-125729 A
PTL 3: JP 2008-543389 A

### SUMMARY OF INVENTION

### Technical Problem

It is recommended to perform blood pressure measurement in a predetermined time period, such as morning and evening, to accurately evaluate a blood pressure state of a user. In a time period other than these time periods, the blood pressure of the user is unstable due to various factors such as exercise, and even if the blood pressure measurement is performed, usefulness for accurate evaluation of the blood pressure state of the user may be low. However, in the above-described related art, there is no disclosure of a means for terminating the notification to prompt blood pressure measurement in consideration of such usefulness.

For example, PTL 1 does not disclose how long to continue the notification that is started when the set time comes. In contrast, for example, it is conceivable to stop the notification when the blood pressure measurement is performed. However, if the user does not perform the blood pressure measurement for a while, it continues to notify the user even when the recommended time period of the blood pressure measurement passes, and the blood pressure measurement that is less useful as described above may be prompted to the user. Further, PTL 2 and 3 do not disclose means for solving such a problem.

In addition, if the notification that does not take into account the above-described usefulness is continued, the appeal power of the notification to prompt the blood pressure measurement may be reduced, and the user's motivation for continuous blood pressure measurement may be reduced.

The present invention, according to an aspect, has been made with reference to such circumstances, and an object of the present invention is to prompt execution of highly useful blood pressure measurement.

### Solution to Problem

The present invention adopts the following configurations to solve the above-described problems. That is, the blood pressure measuring apparatus according to an aspect of the present invention is a blood pressure measuring apparatus for performing blood pressure measurement, the blood pressure measuring apparatus including a notification unit that performs a notification to prompt a user to perform the blood pressure measurement, a time setting unit capable of setting a blood pressure measurement time by an instruction from the user, a storage unit that stores a recommended time period of the blood pressure measurement, and a control unit that performs control to cause the notification unit to start the notification based the blood pressure measurement time set by the time setting unit, and cause the notification unit to stop the notification when the recommend time period passes. The recommended time period of blood pressure measurement is a time period recommended for performing blood pressure measurement in order to accurately evaluate the blood pressure state of the user, and one example thereof is a morning and an evening time period.

In the above-described configuration, when the recommended time period of the blood pressure measurement passes, the notification to prompt the user to perform the blood pressure measurement is stopped even if the blood pressure measurement has not been performed. In this way, it is possible to avoid prompting the user to perform the blood pressure measurement even in a time period in which the usefulness of the blood pressure measurement is low, and to prompt the user to perform blood pressure measurement with high usefulness. Therefore, for example, it is possible to suppress a decrease in motivation of the user for continuous blood pressure measurement.

In the blood pressure measuring apparatus according to the aspect described above, the time setting unit may set the blood pressure measurement time within a range of the recommended time period.

In the blood pressure measuring apparatus according to the aspect described above, a plurality of the recommended time periods may be set. According to this configuration, for a plurality of recommended time periods for which the blood pressure measurement is recommended to accurately evaluate the blood pressure state of the user, it is possible to prevent the notification from continuing even when the recommended time period passes.

In the blood pressure measuring apparatus according to the aspect described above, the plurality of recommended time periods may be a morning time period and an evening time period. According to this configuration, for example, for each recommended time period of morning and evening for each day for which the blood pressure measurement is recommended to accurately evaluate the blood pressure state of the user, it is possible to prevent the notification from continuing even when the recommended time period passes.

In the blood pressure measuring apparatus according to the aspect described above, the notification unit may perform the notification by light emission, and the control unit may stop the notification by stopping the light emission or changing a mode of the light emission.

In the blood pressure measuring apparatus according to the aspect described above, the notification unit may perform the notification by vibration, and the control unit may stop the notification by stopping the vibration or changing a mode of the vibration.

In the blood pressure measuring apparatus according to the aspect described above, the notification unit may perform the notification by screen display, and the control unit may stop the notification by stopping the screen display or changing a mode of the screen display.

In the blood pressure measuring apparatus according to the aspect described above, the notification unit may perform the notification by voice output, and the control unit may stop the notification by stopping the voice output or changing a mode of the voice output.

In the blood pressure measuring apparatus according to the aspect described above, change of the recommended time period by the user may not be possible. According to this configuration, it is possible to avoid a situation in which the user changes the end time of the recommended time period and the notification continues even when the original recommended time period passes.

In the blood pressure measuring apparatus according to the aspect described above, the control unit may change a mode of the notification based on an execution history of the blood pressure measurement.

According to this configuration, since the user can recognize the execution situation of the blood pressure measurement by the mode of the notification, it is possible to support the continuous execution of the blood pressure measurement by the user.

In the blood pressure measuring apparatus according to the aspect described above, the execution history of the blood pressure measurement, may be a history relating to a frequency of the blood pressure measurement. According to this configuration, the lower the frequency of performing the blood pressure measurement, the more often the notification can be made in an aspect that strongly prompts the user to perform the blood pressure measurement, and thus it is possible to support the continuous execution of the blood pressure measurement by the user.

The non-transitory recording medium storing a control program according to an aspect of the present invention is a non-transitory recording medium storing a control program of a blood pressure measuring apparatus for performing a notification to prompt a user to perform blood pressure measurement, the control program causing a computer to execute the steps of starting the notification based on a blood pressure measurement time configurable by an instruction from the user, and stopping the notification when a recommended time period of the blood pressure measurement passes.

### Advantageous Effects of Invention

The present invention provides a technique capable of prompting execution of highly useful blood pressure measurement.

### BRIEF DESCRIPTION OF DRAWINGS

FIG. 1 is a diagram illustrating an example of a blood pressure measuring apparatus 1 to which the present invention is applied.

FIG. 2 is a diagram illustrating an internal configuration of a main body portion 10 in the blood pressure measuring apparatus 1.

FIG. 3 is a flow chart illustrating an example of a notification setting process executed by the blood pressure measuring apparatus 1.

FIG. 4 is a flow chart illustrating an example of a notification process executed by the blood pressure measuring apparatus 1.

FIG. 5 is a diagram illustrating an example of a notification time period set by the blood pressure measuring apparatus 1.

FIG. 6 is a diagram illustrating an example of control of a notification mode in accordance with an execution history of blood pressure measurement.

### DESCRIPTION OF EMBODIMENTS

Hereinafter, embodiments according to an aspect of the present invention will be described with reference to the drawings.

### § 1 Application Example

Blood Pressure Measuring Apparatus 1 to which the Invention is Applied

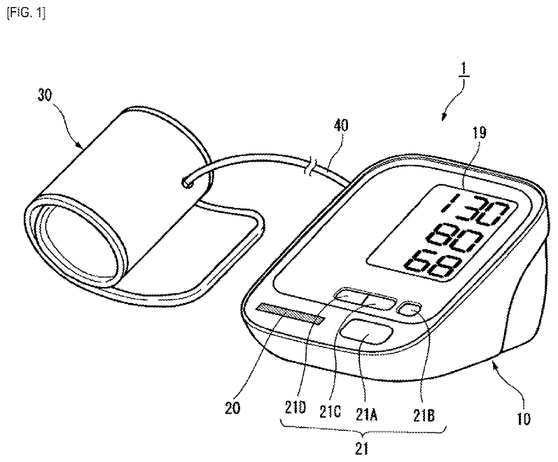

FIG. 1 is a diagram illustrating an example of a blood pressure measuring apparatus 1 to which the present invention is applied. The blood pressure measuring apparatus 1 is a system for measuring blood pressure of a user and outputting a result of the measurement to the user. For example, the blood pressure measuring apparatus 1 includes a main body portion 10, a cuff 30 that can be wrapped around an upper arm of the user, and an air tube 40 that couples the main body portion 10 and the cuff 30. In the example of FIG. 1, the cuff 30 and the main body portion 10 are separate bodies, but the cuff 30 may be integrated with the main body portion 10.

The main body portion 10 includes a display unit 19, an operation unit 21, and a notification unit 20.

The display unit 19 displays various information such as a blood pressure value and a pulse rate. For example, the display unit 19 includes a liquid crystal screen, etc. The operation unit 21 is an operation unit that receives an instruction from the user (measured subject). The configuration of the operation unit 21 will be described later.

The notification unit 20 performs a notification to prompt the user to perform the blood pressure measurement. In the example illustrated in FIG. 1, the notification unit 20 is a light emitting diode (LED) that performs a notification by light emission. When the notification unit 20 is turned on, the user is prompted to perform the blood pressure measurement, and when the notification unit 20 is turned off (stops light emission), the user is not prompted to perform the blood pressure measurement.

The blood pressure measuring apparatus 1 performs control to cause the notification unit 20 to turn on (start the notification) at the blood pressure measurement time, and cause the notification unit 20 to turn off (stop the notification) when the recommended time period of the blood pressure measurement passes. The blood pressure measurement time is, for example, a time configurable by an instruction from the user via the operation unit 21. The recommended time period of the blood pressure measurement is a time period in which the blood pressure measurement is recommended to accurately evaluate the blood pressure state of the user.

The recommended time period is, for example, a morning time period (for example, from 4:00 to 11:59:59) and an evening time period (for example, from 19:00 to 25:59:59). Note that, in this specification, the time is described in 24-hour clock.

That is, a start time of the notification time period in which the blood pressure measuring apparatus 1 performs a notification is configurable by the user. On the other hand, an end time of the notification time period in which the blood pressure measuring apparatus 1 performs a notification coincides with the end time of the recommended time period, and cannot be changed by the user.

As described above, the blood pressure measuring apparatus 1 performs the notification to prompt the user to perform the blood pressure measurement, and stops the notification regardless of the time at which the notification is started when the end time of the recommended time period for performing the blood pressure measurement comes. In this way, it is possible to avoid prompting the user to perform the blood pressure measurement even in a time period in which the usefulness of the blood pressure measurement is low, and to suppress the user to perform blood pressure measurement with low usefulness. Therefore, for example, it is possible to suppress a decrease in motivation of the user for continuous blood pressure measurement.

Note that, since the end time of the notification time period is a time set based on the end time of the recommended time period regardless of the start time of the notification time period, the notification can be stopped in a time period in which the usefulness of blood pressure measurement is low, which is different from a configuration in which the notification is stopped after a certain time elapses from the start of the notification, for example.

In addition, since the end time of the notification time period, that is, the end time of the recommended time period cannot be changed by the user, it is possible to avoid a situation in which the user changes the end time of the notification time period (the end time of the recommended time period) and the notification continues even when the original recommended time period passes.

§ 2 Configuration Example

### Configuration of Blood Pressure Measuring Apparatus 1

### Configuration of Operation Unit 21

The operation unit 21 includes a measurement/stop switch 21A, a memory switch 21B, and an arrow switches 21C and 21D. The measurement/stop switch 21A accepts input of an instruction to turn the power on or off and an instruction to start or end measurement.

The memory switch 21B accepts an instruction to read out information such as blood pressure data stored in the main body portion 10 and display the information on the display unit 19. The arrow switches 21C and 21D accept an instruction to increase or decrease the memory number during the information call.

### Internal Configuration of Main Body Portion 10 in Blood Pressure Measuring Apparatus 1

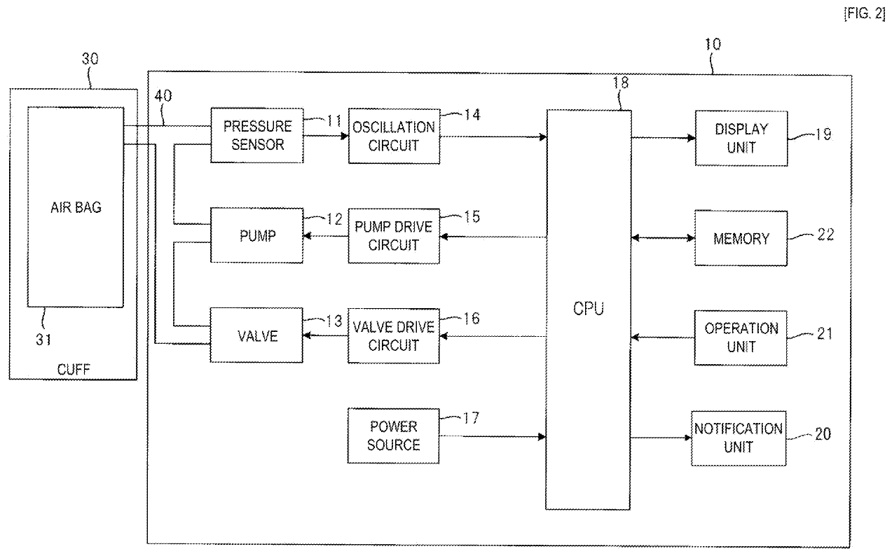

FIG. 2 is a diagram illustrating an internal configuration of the main body portion 10 in the blood pressure measuring apparatus 1.

The cuff 30 includes an air bag 31, and the air tube 40 illustrated in FIG. 1 is coupled to the air bag 31.

The main body portion 10 includes a pressure sensor 11, a pump 12, a valve 13, an oscillation circuit 14, a pump drive circuit 15, a valve drive circuit 16, a power source 17, the display unit 19 illustrated in FIG. 1, the notification unit 20 illustrated in FIG. 1, a central processing unit (CPU) 18, the operation unit 21 illustrated in FIG. 1, and a memory 22. The CPU 18 is an example of the “control unit” according to the present invention. The operation unit 21 is an example of the “time setting unit” according to the present invention. The memory 22 is an example of the “storage unit” according to the present invention.

The pump 12 supplies air to the air bag 31 in order to increase the compression pressure to the target measurement site by the cuff 30.

The valve 13 is opened and closed to expel or enclose the air in the air bag 31.

The pump drive circuit 15 controls driving of the pump 12 based on a control signal supplied from the CPU 18.

The valve drive circuit 16 controls the opening and closing of the valves 13 based on a control signal supplied from the CPU 18.

The pump 12, the valve 13, the pump drive circuit 15, and the valve drive circuit 16 can change the compression pressure to the target measurement site by the cuff 30.

The power source 17 supplies power to each part of the main body portion 10.

The CPU 18 integrally controls the entire main body portion 10 and performs various arithmetic processing. The notification by the notification unit 20 described above is controlled by the CPU 18.

The pressure sensor 11 is a pressure sensor coupled to the air tube 40, and is a capacitive pressure sensor as an example. The capacitive pressure sensor is one that changes the capacitance value in accordance with the detected pressure.

The oscillation circuit 14 oscillates based on the capacitance value of the pressure sensor 11, and outputs a signal corresponding to the capacitance value to the CPU 18. The CPU 18 detects the pressure of the cuff 30 (cuff pressure) by converting the signal output from the oscillation circuit 14 into a pressure value.

The memory 22 includes a non-transitory recording medium for storing programs and a read only memory (ROM) for storing data for causing the CPU 18 to perform a predetermined operation, a random access memory (RAM) as a work memory, and a flash memory for storing measured blood pressure data, etc. The recommended time period described above is, for example, stored in the ROM, the flash memory, and the like of the memory 22.

### Processing of Blood Pressure Measuring Apparatus 1

The processing of the blood pressure measuring apparatus 1 described below is mainly executed by the CPU 18, for example, by the CPU 18 reading and executing the control program stored in the ROM of the memory 22.

### Notification Setting Process by Blood Pressure Measuring Apparatus 1

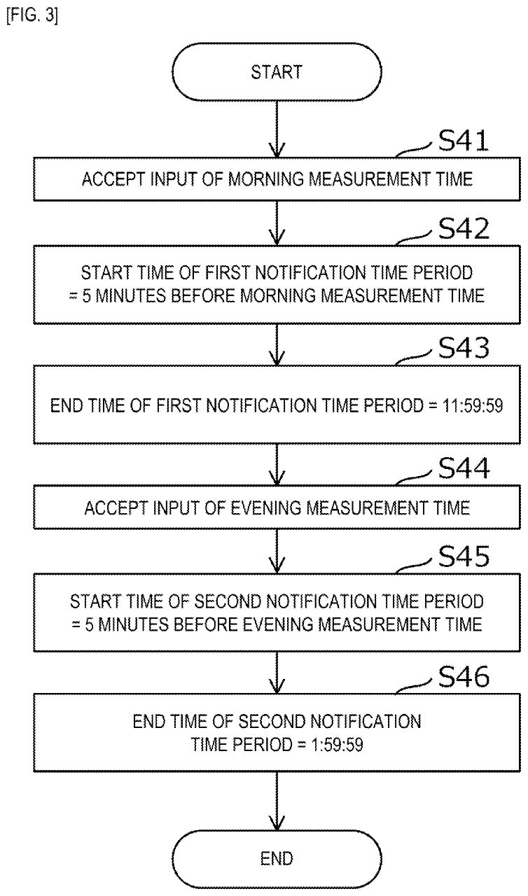

FIG. 3 is a flow chart illustrating an example of a notification setting process executed by the blood pressure measuring apparatus 1. The blood pressure measuring apparatus 1 executes the process illustrated in FIG. 3, for example, as a process for setting the notification time period for performing the above-described notification.

First, the blood pressure measuring apparatus 1 accepts the input of the morning measurement time by the operation unit 21 from the user (step S41). The morning measurement time is a time at which the user performs blood pressure measurement in the morning recommended time period. The morning recommended time period is, for example, a time period from 4:00 to 11:59:59.

Next, the blood pressure measuring apparatus 1 sets the start time of a first notification time period, which is the morning notification time period in which the above notification is performed, to 5 minutes before the morning measurement time accepted by step S41 (step S42). Furthermore, the blood pressure measuring apparatus 1 also sets the end time of the first notification time period to 11:59:59, which is the end time of the morning recommended time period (step S43). By steps S41 to S43, the first notification time period is set.

Next, the blood pressure measuring apparatus 1 accepts the input of the evening measurement time by the operation unit 21 from the user (step S44). The evening measurement time is a time at which the user performs blood pressure measurement in the evening recommended time period. The evening recommended time period is, for example, a time period from 19:00 to 25:59:59.

Next, the blood pressure measuring apparatus 1 sets the start time of a second notification time period, which is the evening notification time period in which the above notification is performed, to 5 minutes before the evening measurement time accepted by step S44 (step S45). Furthermore, the blood pressure measuring apparatus 1 also sets the end time of the second notification time period to 1:59:59 (25:59:59), which is the end time of the evening notification time period (step S46). By steps S44 to S46, the second notification time period is set.

As illustrated in FIG. 3, the start time of the notification time period including the first notification time period and the second notification time period is set by input from the user. On the other hand, the end time of the notification time period is fixedly set based on the recommended time period and cannot be set by the user.

### Notification Process by Blood Pressure Measuring Apparatus 1

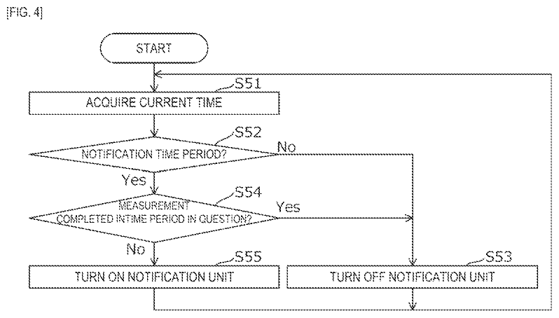

FIG. 4 is a flow chart illustrating an example of a notification process executed by the blood pressure measuring apparatus 1. The blood pressure measuring apparatus 1 executes the process illustrated in FIG. 4, for example, as a process for performing the above-described notification.

First, the blood pressure measuring apparatus 1 acquires a current time (step S51). For example, the blood pressure measuring apparatus 1 acquires the current time based on an internal clock included in the blood pressure measuring apparatus 1. Next, the blood pressure measuring apparatus 1 determines whether the current time acquired by step S51 is the time in the notification time period (step S52). For example, the blood pressure measuring apparatus 1 determines whether the current time is any time of the first notification time period and the second notification time period illustrated in FIG. 3.

In step S52, when the current time is not the time in the notification time period (step S52: No), the blood pressure measuring apparatus 1 performs control of turning off the notification unit 20 (step S53), and returns to step S51. In particular, in step S53, the blood pressure measuring apparatus 1 performs control to turn off the notification unit 20 when the notification unit 20 is turned on, and to maintain the turn-off of the notification unit 20 when the notification unit 20 is turned off.

In step S52, when the current time is a time in the notification time period (step S52: Yes), the blood pressure measuring apparatus 1 determines whether the blood pressure measurement by the blood pressure measuring apparatus 1 has been performed (measurement completed) in the time period in question (step S54). Note that the time period in question is a notification time period including the current time, and does not include a notification time period in the previous day, and the like before the notification time period including the current time.

In step S54, when the blood pressure measurement is performed (step S54: Yes), the blood pressure measuring apparatus 1 transitions to step S53. When the blood pressure measurement has not been performed (step S54: No), the blood pressure measuring apparatus 1 performs control to turn on the notification unit 20 (step S55), and returns to step S51. In particular, in step S55, the blood pressure measuring apparatus 1 performs control to turn on the notification unit 20 when the notification unit 20 is turned off, and to maintain the turn-on of the notification unit 20 when the notification unit 20 is turned on.

### Control Program

The control program of the blood pressure measuring apparatus 1 is stored in a non-transitory computer readable storage medium. Such a “computer-readable storage medium” includes, for example, an optical medium such as a compact disc-ROM (CD-ROM), a magnetic storage medium such as a universal serial bus (USB) memory stick or a memory card, etc. Such a program can also be provided by downloading via a network such as the Internet.

### § 3 Operation Example

Notification Time Period Set by Blood Pressure Measuring Apparatus 1

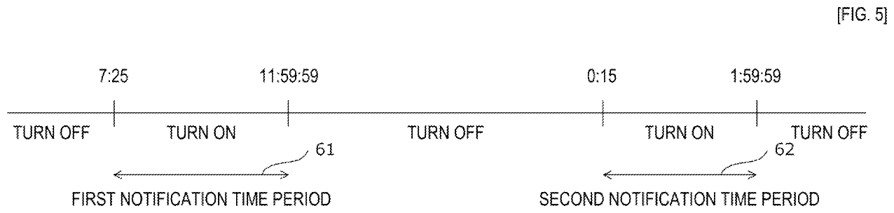

FIG. 5 is a diagram illustrating an example of a notification time period set by the blood pressure measuring apparatus 1. The first notification time period 61 illustrated in FIG. 5 is an example of the first notification time period set by steps S41 to S43 illustrated in FIG. 3. The second notification time period 62 illustrated in FIG. 5 is an example of the second notification time period set by steps S44 to S46 illustrated in FIG. 3.

In the example illustrated in FIG. 5, description will be given for a case where 7:30 is input as the morning measurement time in step S41 illustrated in FIG. 3 and 0:20 is input as the evening measurement time in step S44 illustrated in FIG. 3.

In this case, the first notification time period 61 is a time period in which the start time is 7:25, which is 5 minutes before 7:30, and the end time is 11:59:59. The second notification time period 62 is a time period in which the start time is 0:15, which is 5 minutes before 0:20, and the end time is 1:59:59.

If the user does not perform the blood pressure measurement with the blood pressure measuring apparatus 1, the blood pressure measuring apparatus 1 performs control to turn on the notification unit 20 (LED) in the first notification time period 61 and the second notification time period 62, and turn off the notification unit 20 in other time periods, as illustrated in FIG. 5.

Although not illustrated, when the user performs the blood pressure measurement with the blood pressure measuring apparatus 1 in the first notification time period 61, the blood pressure measuring apparatus 1 performs control to turn off the notification unit 20 during the remaining period of the first notification time period 61. Similarly, when the user performs the blood pressure measurement with the blood pressure measuring apparatus 1 in the second notification time period 62, the blood pressure measuring apparatus 1 performs control to turn off the notification unit 20 during the remaining period of the second notification time period 62.

### § 4. Modified Examples

Although the embodiments of the present invention have been described in detail above, the above description is merely an example of the present invention in all respects. Various modifications and changes may be made without departing from the scope of the invention. For example, the following changes are possible. Note that, in the following, the same reference numerals are used for components that are the same as those of the above-described embodiment, and descriptions thereof are omitted as appropriate. The following modified examples can be combined as appropriate.

### MODIFIED EXAMPLE 1

Control of Notification Mode according to Execution History of Blood Pressure Measurement

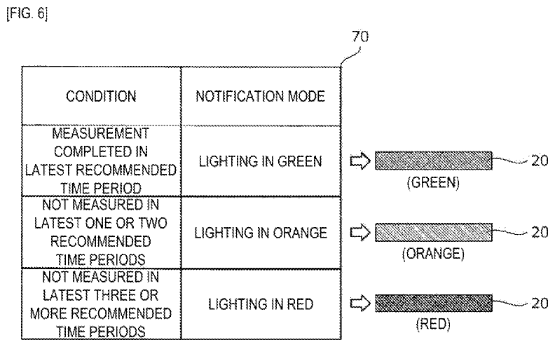

FIG. 6 is a diagram illustrating an example of control of a notification mode in accordance with an execution history of blood pressure measurement. The blood pressure measuring apparatus 1 may change modes of the above-described notification based on an execution history of the blood pressure measurement by the blood pressure measuring apparatus 1. For example, the blood pressure measuring apparatus 1 stores the execution history of the blood pressure measurement by the blood pressure measuring apparatus 1 on the memory 22, etc. The execution history of the blood pressure measurement includes, for example, information indicating the date and time when the blood pressure measurement was performed.

Additionally, a control table 70 illustrated in FIG. 6 is stored in advance in the memory 22 of the blood pressure measuring apparatus 1. In the control table 70, the mode of the notification of the notification unit 20 is associated with each condition related to the execution history of the blood pressure measurement.

For example, the control table 70 indicates that the notification unit 20 is lit in green when the blood pressure measurement has been performed (measurement completed) by the blood pressure measuring apparatus 1 in the latest recommended time period. Note that the latest recommended time period is the newest recommended time period of the recommended time periods before the current time.

Furthermore, the control table 70 indicates that the notification unit 20 is lit in orange when the blood pressure measurement is not performed by the blood pressure measuring apparatus 1 in the latest one or two recommended time periods. In addition, the control table 70 indicates that the notification unit 20 is lit in red when the blood pressure measurement is not performed by the blood pressure measuring apparatus 1 in the latest three or more recommended time periods.

For example, in step S55 illustrated in FIG. 4, the blood pressure measuring apparatus 1 controls the lighting color of the notification unit 20 based on the execution history of blood pressure measurement and the control table 70. As an example, assuming that a certain day is the first day, when blood pressure measurement is performed in the evening recommended time period of the first day, the notification unit 20 lights up in green in the notification time period related to the morning recommended time period of the second day. Then, when the blood pressure measurement is not performed in the morning recommended time period of the second day, the notification unit 20 lights up in orange in the notification time period related to the evening recommended time period of the second day.

Thus, it is possible to prompt the user to perform blood pressure measurement and to cause the user to recognize the execution situation of the blood pressure measurement. Accordingly, it is possible to assist in performing continuous blood pressure measurement by the user. For example, as illustrated in the example illustrated in FIG. 6, it is possible to more strongly prompt the user to perform blood pressure measurement by performing a notification using a more conspicuous color as the situation continues in which the blood pressure measurement is not performed in the recommended time period.

In the example illustrated in FIG. 6, the control of changing the lighting color of the notification unit 20 is described as the control to change the mode of the notification by the notification unit 20, but the control of changing the mode of the notification by the notification unit 20 is not limited thereto. For example, the control of changing the mode of the notification by the notification unit 20 may be the control to change the brightness of the lighting by the notification unit 20, or may be the control to change the lighting pattern such as lighting and blinking of the notification unit 20.

Further, the condition regarding the execution history of the blood pressure measurement is not limited to the example illustrated in FIG. 6. For example, the condition regarding the execution history of the blood pressure measurement may be a condition of the latest consecutive number of days on which the blood pressure measurement is not performed in the morning or in the evening. As described above, the condition regarding the execution history of the blood pressure measurement can be various conditions regarding the execution frequency of the blood pressure measurement.

The condition regarding the execution history of the blood pressure measurement is not limited to the condition regarding the execution frequency of the blood pressure measurement. For example, the condition regarding the execution history of the blood pressure measurement may be a condition regarding whether the blood pressure measurement has been performed in the latest recommended time period, a condition regarding whether the blood pressure measurement has been performed in at least one of the recommended time periods in the morning and evening of the previous day, etc. Alternatively, the condition regarding the execution history of the blood pressure measurement may be a condition of the cumulative number of times of the blood pressure measurement performed so far in the blood pressure measuring apparatus 1.

### MODIFIED EXAMPLE 2

### Modified Example of Notification Stop Method

The method of configuring the notification unit 20 by an LED, etc. and turning off the notification unit 20 has been described as the method of stopping the notification by the notification unit 20. However, the method of stopping the notification by the notification unit 20 is not limited thereto.

For example, the blood pressure measuring apparatus 1 may stop the notification by changing the mode of light emission by the notification unit 20, such as reducing the luminance of light emission by the notification unit 20, changing the color of light emission by the notification unit 20 to an inconspicuous color, or shielding the light emitting unit of the notification unit 20 with a shutter, etc. Alternatively, the blood pressure measuring apparatus 1 may blink the notification unit 20 as a notification to prompt the user to perform the blood pressure measurement, and stop the notification by switching the notification unit 20 from blinking to lighting.

### MODIFIED EXAMPLE 3

### Modified Example of Notification Method

As a method of notification by the notification unit 20, a method of performing a notification by light emission of the notification unit 20 configured by an LED, etc. has been described, but the method of notification by the notification unit 20 is not limited thereto.

For example, the notification unit 20 may perform a notification by vibration using a vibrator. In this case, the blood pressure measuring apparatus 1 stops the notification by stopping the vibration of the notification unit 20 or changing the mode of the vibration of the notification unit 20.

Alternatively, the notification unit 20 may perform a notification by screen display using a display. In this case, the blood pressure measuring apparatus 1 stops the notification by stopping the screen display of the notification unit 20 or changing the mode of the screen display of the notification unit 20.

Alternatively, the notification unit 20 may perform a notification by voice output using a speaker. In this case, the blood pressure measuring apparatus 1 stops the notification by stopping the voice output of the notification unit 20 or changing the mode of the voice output of the notification unit 20.

In addition, regarding the notification unit 20, a case where the notification unit 20 itself is configured by an output apparatus such as an LED has been described, but the notification unit 20 may perform a notification by controlling an external output apparatus. For example, the notification unit 20 may be capable of communicating with an information terminal such as a smartphone that is owned by a user, and a notification may be performed by controlling the light emission, vibration, screen display, or voice output of the information terminal.

Further, the notification unit 20 may perform a notification by combining a plurality of notification methods among the above-described notification methods.

### MODIFIED EXAMPLE 4

### Modified Example of End Time of Notification Time Period

Although a case has been described in which the end time of the notification time period is a time that is set regardless of the start time of the notification time period, the end time of the notification time period may be a time that is linked to the start time of the notification time period as long as the time is based on the recommended time period for performing the blood pressure measurement. For example, the default value of the end time of the notification time period may be the end time of the recommended time period, and the end time of the notification time period may be a time slightly (for example, several seconds or several minutes) shifted from the default value according to the start time of the notification time period.

In addition, although a case has been described in which the end time of the notification time period cannot be changed by the user, the end time of the notification time period may be changed by the user as long as the end time is a time based on the recommended time period for performing the blood pressure measurement. For example, the default value of the end time of the notification time period may be the end time of the recommended time period, and the end time of the notification time period may be a time slightly (for example, several seconds or several minutes) shifted from the default value according to setting by the user. Alternatively, the end time of the notification time period may be a time selected by the user from a plurality of times close to the end time of the recommended time period.

While various embodiments have been described with reference to the drawings, needless to say, the present invention is not limited to such examples. It will be apparent to those skilled in the art that various changes and modifications can be made within the scope of the claims, and it is understood that these are naturally belong within the technical scope of the present invention. Further, each of the components of the above-described embodiments may be combined as desired within a range that does not depart from the spirit of the present invention.

Note that the present application is based on Japanese Patent Application filed on Oct. 29, 2019 (Japanese Patent Application No. 2019-196628), the contents of which are incorporated herein by reference.

### REFERENCE NUMERALS LIST

1 Blood pressure measuring apparatus
10 Main body portion
11 Pressure sensor
12 Pump
13 Valve
14 Oscillation circuit
15 Pump drive circuit
16 Valve drive circuit
17 Power source
18 CPU
19 Display unit
20 Notification unit
21 Operation unit
21A measurement/stop switch
21B Memory switch
21C, 21D Arrow switch
22 Memory
30 Cuff
31 Air bag
40 Air tube
61 First notification time period
62 Second notification time period
70 Control table

1. A blood pressure measuring apparatus for performing blood pressure measurement, the blood pressure measuring apparatus comprising:
a notification unit that performs a notification to prompt a user to perform the blood pressure measurement;
a time setting unit capable of setting a blood pressure measurement time by an instruction from the user;
a storage unit that stores a recommended time period of the blood pressure measurement, the recommended time period being unchangeable by the user; and
a control unit that performs control to cause the notification unit to start the notification based the blood pressure measurement time set by the time setting unit, and cause the notification unit to stop the notification when the recommend time period passes.

2. The blood pressure measuring apparatus according to claim 1, wherein
the time setting unit sets the blood pressure measurement time within a range of the recommended time period.

3. The blood pressure measuring apparatus according to claim 1, wherein
a plurality of the recommended time periods are set.

4. The blood pressure measuring apparatus according to claim 3, wherein
the plurality of recommended time periods are a morning time period and an evening time period.

5. The blood pressure measuring apparatus according to claim 1, wherein
the notification unit performs the notification by light emission, and
the control unit stops the notification by stopping the light emission or changing a mode of the light emission.

6. The blood pressure measuring apparatus according to claim 1, wherein
the notification unit performs the notification by vibration, and
the control unit stops the notification by stopping the vibration or changing a mode of the vibration.

7. The blood pressure measuring apparatus according to claim 1, wherein
the notification unit performs the notification by screen display, and
the control unit stops the notification by stopping the screen display or changing a mode of the screen display.

8. The blood pressure measuring apparatus according to claim 1, wherein
the notification unit performs the notification by voice output, and
the control unit stops the notification by stopping the voice output or changing a mode of the voice output.

9. The blood pressure measuring apparatus according to claim 1, wherein
the control unit changes a mode of the notification based on an execution history of the blood pressure measurement.

10. The blood pressure measuring apparatus according to claim 9, wherein
the execution history of the blood pressure measurement is a history relating to a frequency of the blood pressure measurement.

11. A non-transitory recording medium storing a control program of a blood pressure measuring apparatus for performing a notification to prompt a user to perform blood pressure measurement, the control program causing a computer to execute the steps of:
starting the notification based on a blood pressure measurement time configurable by an instruction from the user; and
stopping the notification when a recommended time period of the blood pressure measurement passes, the recommended time period being unchangeable by the user.
